(19) United States
(12) Patent Application Publication
(10) Pub. No.: US 2026/0002928 A1
(43) Pub. Date: Jan. 1, 2026

(54) DROPOUT DETECTION IN CONTINUOUS ANALYTE MONITORING DATA DURING DATA EXCURSIONS

(71) Applicant: ABBOTT DIABETES CARE INC., Alameda, CA (US)

(72) Inventors: Junli Ou, Pleasanton, CA (US); Erwin Satrya Budiman, Fremont, CA (US)

(21) Appl. No.: 19/322,384

(22) Filed: Sep. 8, 2025

Related U.S. Application Data
(63) Continuation of application No. 17/171,768, filed on Feb. 9, 2021, now abandoned.
Application No. 17/171,768 is a continuation of application No. 16/850,937, filed on Apr. 16, 2020, now Pat. No. 10,942,164.
Application No. 16/850,937 is a continuation of application No. 16/504,986, filed on Jul. 8, 2019, now Pat. No. 10,656,139.
Application No. 16/504,986 is a continuation of application No. 16/193,881, filed on Nov. 16, 2018, now Pat. No. 10,345,291.
Application No. 16/193,881 is a continuation of application No. 14/424,026, filed on Feb. 25, 2015, now Pat. No. 10,132,793.
(60) Provisional application No. 61/695,164, filed on Aug. 30, 2012.

(51) Int. Cl. G01N 33/49; A61B 5/145; G06F 16/901; G16H 10/60; G16Z 99/00

(52) U.S. Cl. CPC G01N 33/49; A61B 5/14532; G06F 16/9024; G16H 10/60; G16Z 99/00; A61B 2560/0276; G06F 2218/16

(57) ABSTRACT

Methods, devices, and systems are provided for identifying dropouts in analyte monitoring system sensor data including segmenting sensor data into a plurality of time series wherein each time series is associated with a different instance of a repeating event, selecting a first time series to analyze for dropouts from the plurality of time series; comparing the selected first time series to a second time series among the plurality of time series, determining whether the selected first time series includes a portion that is more than a predefined threshold lower than a corresponding portion of the second time series, and displaying, on a computer system display, an indication that the selected first time series includes a dropout if the selected first time series includes a portion that is more than the predefined threshold lower than the corresponding portion of the second time series.

### CROSS-REFERENCE TO RELATED APPLICATIONS

This application is a continuation of U.S. patent application Ser. No. 17/171,768 filed Feb. 9, 2021, which is a continuation of U.S. patent application Ser. No. 16/850,937 filed Apr. 16, 2020, now U.S. Pat. No. 10,942,164, which is a continuation of U.S. patent application Ser. No. 16/504,986 filed Jul. 8, 2019, now U.S. Pat. No. 10,656,139, which is a continuation of U.S. patent application Ser. No. 16/193,881 filed Nov. 16, 2018, now U.S. Pat. No. 10,345,291, which is a continuation of U.S. patent application Ser. No. 14/424,026 filed Feb. 25, 2015, now U.S. Pat. No. 10,132,793, which claims priority to PCT Application No. PCT/US2013/055883 filed Aug. 20, 2013, which claims priority to U.S. Provisional Patent Application No. 61/695,164, filed on Aug. 30, 2012, entitled “Dropout Detection in Continuous Analyte Monitoring Data During Data Excursions”, the disclosures of each of which are incorporated herein by reference in their entirety for all purposes.

### BACKGROUND

The detection of the concentration level of glucose or other analytes in certain individuals may be vitally important to their health. For example, the monitoring of glucose levels is particularly important to individuals with diabetes or pre-diabetes. People with diabetes may need to monitor their glucose levels to determine when medication (e.g., insulin) is needed to reduce their glucose levels or when additional glucose is needed.

Devices have been developed for automated in vivo monitoring of analyte time series characteristics, such as glucose levels, in bodily fluids such as in the blood stream or in interstitial fluid. Some of these analyte level measuring devices are configured so that at least a portion of the devices are positioned below a skin surface of a user, e.g., in a blood vessel or in the subcutaneous tissue of a user. As used herein, the term analyte monitoring system is used to refer to any type of in vivo monitoring system that uses a sensor disposed with at least a portion subcutaneously to measure and store sensor data representative of analyte concentration levels automatically over time. Analyte monitoring systems include both (1) systems such as continuous glucose monitors (CGMs) which transmit sensor data continuously or at regular time intervals (e.g., once per minute) to a processor/display unit and (2) systems that transfer stored sensor data in one or more batches in response to a request from a processor/display unit (e.g., based on an activation action and/or proximity using a near field communications protocol).

In some cases, analyte monitoring systems have been found to occasionally provide false low readings for relatively short periods (e.g., non-zero-mean signal artifacts). These false low readings, referred to as “dropouts,” are distinct from a situation where no reading at all is provided. When no data at all is provided, an analyte monitoring system can easily detect that there is a problem because there simply is no signal from the sensor. In the case of a dropout however, there is still a signal and the data may appear to be correct but in fact, the data is temporarily incorrect. In a CGM for example, such false data can trigger an unnecessary low blood sugar (e.g., hypoglycemia event) false alarm. Thus, what is needed are systems, methods, and apparatus to reliably determine when a dropout has occurred in analyte monitoring system sensor data.

### SUMMARY

The present invention provides systems, methods, and apparatus that allow a user to analyze a collection of analyte monitoring system sensor data to identify dropouts. By improving dropout identification capabilities, the present invention enables researchers to better understand dropout characteristics and thereby potentially develop a detection and correction algorithm that, for example, could be incorporated into a future analyte monitor system to detect and correct for dropouts as they are happening. In addition, improved dropout identification can facilitate research efforts to mitigate these errors and to provide a basis to compare sensor designs. Better dropout recognition may also allow healthcare providers and patients to calibrate analyte monitor system alarm thresholds more accurately to reduce false alarms. Embodiments of the present invention segments analyte monitoring system sensor data into time series associated with repeating events, such as meals, that cause analyte levels to vary over time. Using one or more of curve smoothing, time dilation, and dynamic range scaling techniques to normalize the data for comparison, two or more time series that correspond to the same repeating event (e.g., breakfast) on different days are compared. If a time series includes a period with an anomalous low level compared with the same period in another corresponding time series, the period is identified as a dropout.

Some embodiments of the present disclosure include computer-implemented methods of identifying a dropout in analyte monitoring system sensor data. The methods include receiving a signal representative of sensor data from an analyte monitoring system related to an analyte level of a patient measured over time; storing the sensor data in a computer system storage device; segmenting the sensor data into a plurality of time series wherein each time series is associated with a different instance of a repeating event; selecting a first time series to analyze for dropouts from the plurality of time series; comparing the selected first time series to a second time series among the plurality of time series; determining whether the selected first time series includes a portion that is more than a predefined threshold lower than a corresponding portion of the second time series; and displaying, on a computer system display, an indication that the selected first time series includes a dropout if the selected first time series includes a portion that is more than the predefined threshold lower than the corresponding portion of the second time series. The invention also includes a computer system and a computer program product for identifying a dropout in analyte monitoring system sensor data. Numerous other aspects and embodiments are provided. Other features and aspects of the present invention will become more fully apparent from the following detailed description, the appended claims, and the accompanying drawings.

### BRIEF DESCRIPTION OF THE DRAWINGS

FIG. 1 depicts an example graph of analyte monitoring system sensor data plotted over time in accordance with some embodiments of the present invention.

FIG. 2 depicts an example graph of segmented and overlaid analyte monitoring system sensor data plots in accordance with some embodiments of the present invention.

FIGS. 3A to 3D depict an example graph sequence of two smoothed time series plots of sensor data corresponding to the same repeating event occurring on different days, one having been time dilated and dynamic range adjusted, in accordance with some embodiments of the present invention.

FIGS. 4A to 4C depict an example graph sequence of two smoothed time series plots of sensor data, one having been time dilated in accordance with some embodiments of the present invention.

FIG. 5 depicts a flow chart illustrating an example method in accordance with some embodiments of the present invention.

### DETAILED DESCRIPTION

The present invention provides systems, methods, and apparatus to identify dropouts in sensor data from an analyte monitoring system, such as, for example, any type of in vivo monitoring system that uses a sensor disposed with at least a portion subcutaneously to measure and store sensor data representative of analyte concentration levels automatically over time. Analyte monitoring systems may include CGMs which are programmed to transmit sensor data according to a predetermined transmission schedule, continuously, or at regular time intervals to a processor/display unit and systems that transfer stored sensor data in one or more batches in response to a request from a processor/display unit, i.e., not according to a predetermined transmission schedule. Without requiring a patient to wear two or more analyte sensors, the present invention is operable to identify dropouts in data from a single analyte sensor. According to some embodiments of the present invention, data representative of a patient's monitored analyte concentration level (herein referred to as “sensor data”) previously captured over a period of time (e.g., two weeks) is segmented into time series representative of sensor data reflecting the effects of a meal or other repeating event that causes data “excursions” from a stable analyte concentration level. Several time series representing repeating events, for example, a meal, or an activity occurring at a fixed time of day, may be identified for comparative analysis. In some embodiments, event time markers, either manually input into the system or automatically determined from meal start estimates (without user intervention), may be used to identify the beginning and end of a time series. Once two or more time series have been identified as corresponding to different instances of the same repeating event, the corresponding time series can be analyzed to detect dropouts. In some embodiments of the present invention, corresponding time series are compared to identify low analyte level differences that represent dropouts. However, since a patient's analyte levels do not follow an identical pattern each day and since excursion events such as meals are longer or shorter on different days, data modulation algorithms or techniques may be employed to normalize data (time series normalized data) or otherwise process or transform data to match it to other time series data for comparison. Harmonizing sets of un-harmonized sensor data time series to each other enables similar data sets that are not exactly identical to be compared to each other. Time series modulation algorithms or techniques may employ one or more of time series curve smoothing, dynamic range matching, and time dilation, to aid in the comparison of the corresponding time series.

The invention may be applied to any analyte concentration level determination system that may exhibit or at least be suspected of exhibiting, or that may be susceptible to, dropouts. Embodiments of the invention are described primarily with respect to continuous glucose monitoring devices and systems but the present invention may be applied to other analytes and analyte characteristics, as well as data from measurement systems that transmit sensor data from a sensor unit to another unit such as a processing or display unit in response to a request from the other unit. For example, other analytes that may be monitored include, but are not limited to, acetyl choline, amylase, bilirubin, cholesterol, chorionic gonadotropin, creatine kinase (e.g., CK-MB), creatine, DNA, fructosamine, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times. The present invention also provides numerous additional embodiments.

Embodiments of the present invention may include a programmed computer system adapted to receive and store data from an analyte monitoring system. The computer system may include one or more processors for executing instructions or programs that implement the methods described herein. The computer system may include memory and persistent storage devices to store and manipulate the instructions and sensor data received from the analyte monitoring system. The computer system may also include communications facilities (e.g., wireless and/or wired) to enable transfer of the sensor data from the analyte monitoring system to the computer. The computer system may include a display and/or output devices for identifying dropouts in the sensor data to a user. The computer system may include input devices and various other components (e.g., power supply, operating system, clock, etc.) that are typically found in a conventional computer system. In some embodiments, the computer system may be integral to the analyte monitoring system. For example, the computer system may be embodied as a handheld or portable receiver unit within the analyte monitoring system.

The various methods described herein for performing one or more processes also described herein may be embodied as computer programs (e.g., computer executable instructions and data structures) developed using an object oriented programming language that allows the modeling of complex systems with modular objects to create abstractions that are representative of real world, physical objects and their interrelationships. However, any practicable programming language and/or techniques may be used. The software for performing the inventive processes, which may be stored in a memory or storage device of the computer system described herein, may be developed by a person of ordinary skill in the art based upon the present disclosure and may include one or more computer program products. The computer program products may be stored on a computer readable medium such as a server memory, a computer network, the Internet, and/or a computer storage device.

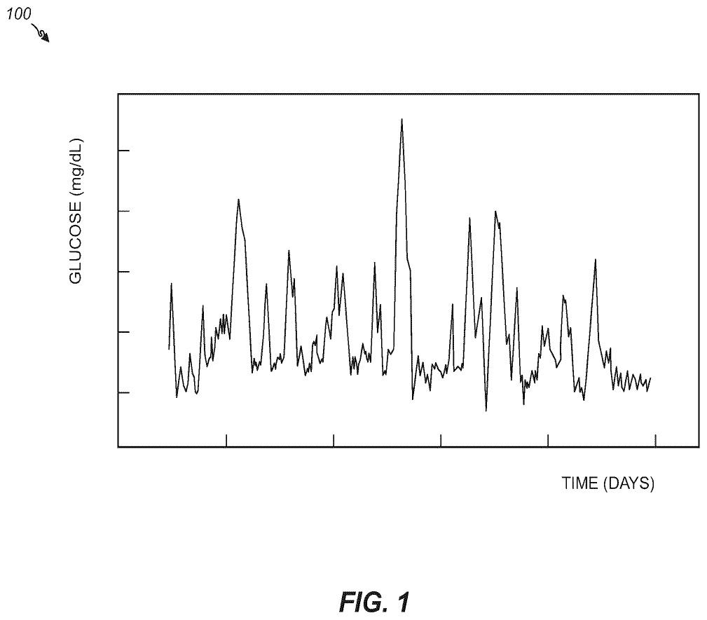

Turning to FIG. 1, a graph 100 of glucose concentration level (mg/dL) over time (days) depicting an example of sensor data generated by an analyte monitoring system is shown. The graph 100 plots a patient's glucose level over the lifetime of the analyte monitoring system's sensor. For example, the particular example graph 100 shown represents data collected by an analyte monitoring system over five days. The variations in the patient's glucose level are referred to as excursions and the excursions may be associated with events in the patient's daily activities. In particular, meals cause excursions as the in-take of food affects the patient's glucose level. However, looking at the graph 100 in FIG. 1, it may not be easy to identify meal excursions because any eating patterns of the patient are not obvious when the data is plotted as one long sequence.

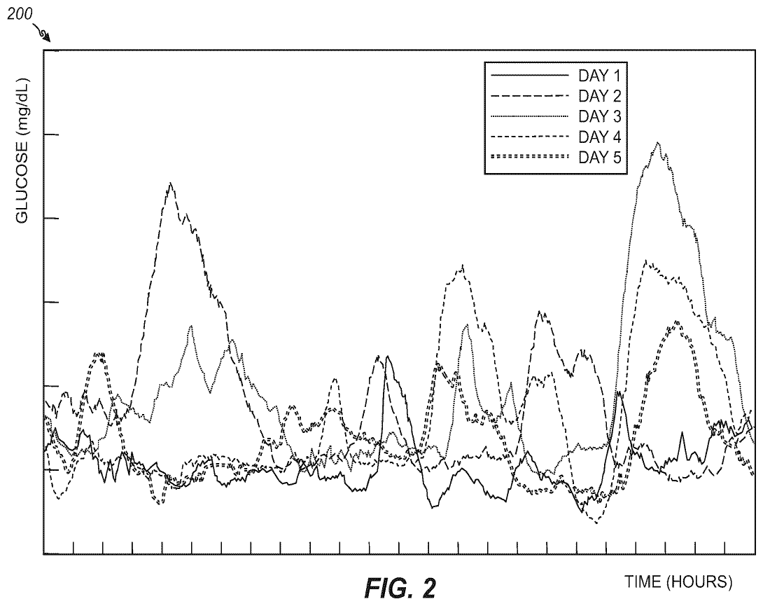

According to embodiments of the present invention, the sensor data may be segmented by the computer system into daily plots that are overlaid to reveal the patient's behavior patterns as shown in the graph 200 of FIG. 2. The overlaid plots may thus be used to identify meal excursions in the data. For example, in the particular graph 200 shown, the grouping of peaks at about 2 PM and about 8 PM suggest that these peaks correspond to daily meals consumed at roughly the same time each day wherein the 2 PM peaks correspond to lunch and the 8 PM peaks correspond to dinner.

In some embodiments, the plots of the sensor data may be, for example, sliding-average smoothed to remove “noise” and to facilitate easier comparison of the plots. In an unweighted sliding-average smoothing, each data point of the plot is replaced with the average of “m” adjacent points, where “m” is a positive integer referred to as the “smoothing width” or “smoothing window” in the case of a data plot over time as here. In some embodiments, various different smoothing windows (e.g., 15 minutes, 20 minutes, 30 minutes, etc.) may be tried by the computer system to find acceptable smoothening results while not significantly distorting features associated with true glucose excursions. For example, the smallest smoothing window that removes most of the jagged points on the curve without changing the overall shape of the curve may be selected. In some embodiments, other methods of smoothing may be used.

Once the overlaid daily plots are smoothed, they may be segmented into time series that correspond to repeating events (e.g., meals). A window of time may be defined where two or more time series of sensor data (e.g., x1, x2, x3, . . . , xN) from the same patient is to be compared. For example, the start of the time window for a repeating event that occurred on the last day of the sensor data may be determined using the latest meal marker (e.g., breakfast, lunch, dinner). This meal marker may be input by the user or determined by a meal start estimator. Details describing obtaining a meal marker from a meal start estimator may be found in U.S. Patent Application No. 61/582,209, filed Dec. 30, 2011, entitled “Method and Apparatus for Determining Medication Dose Information,” which is hereby incorporated by reference. The end of the time window for a repeating event that occurred on the last day of the sensor data may be the latest available data from the sensor, or a time at which a new meal marker has been identified/provided. The start of the time window for a repeating event that occurred on previous days may be determined by meal markers from the appropriate prior days of the same type (e.g., breakfast matched with breakfast, or generic meal matched to the nearest time-of-day as provided by the analyte monitoring system's clock). Thus, using the smoothed, overlaid sensor data, the computer system may be programmed to identify two or more corresponding time series of sensor data that represent excursions resulting from the same repeating event occurring on different days.

Assuming the computer system identifies N pre-smoothed time series, there will be N−1 time series to compare against the x1 time series. Because patients' analyte levels do not follow an identical pattern day to day, and since each breakfast, lunch, or dinner may last longer or shorter than prior days' breakfast, lunch, or dinner, it may be helpful to perform some time dilation (or time stretching) functions on the sensor data to facilitate comparison between two different time series. Therefore, for each of the N−1 comparisons (e.g., x1 vs. x2; x1 vs. x3; . . . x1 vs. xN−1), the computer system may perform the following steps.
a. Set y1 equal to the x1 time series and define y2 to be the next time series to compare against (e.g., x2, x3, . . . , or xN−1).
b. Define a finite set of time dilation (Ta) parameters.
   For example: T_{d}={5/10, 6/10, 7/10, 8/10, 9/10, 1, 10/9, 10/8, 10/7, 10/6, 10/5}
   Where a T_{d} range of a factor of two is selected as sufficient to cover the anticipated time span of variation between different excursions. Other values may be used.
c. For each T_{d} value:
   i. Stretch or compress y2 by the T_{d} value.
      For example, for T_{d}=5/10=1/2, the time series y2 is compressed to be half as short as the original. For T_{d}=10/5=2, the time series y2 is stretched to be twice as long as the original.
   ii. Compute y2′, where the values are interpolated such that the time series y2′ has the same sample time as y1. A linear interpolation method or any other practicable method of interpolation may be used.
   iii. Compute the cross correlation between y1 and y2′.
   iv. Repeat by using the next T_{d} value in the set.
d. Find the T_{d} value that results in the highest cross-correlation between y1 and y2′. The T_{d} value that results in the highest cross-correlation between y1 and y2′ is the best time dilation adjustment.
e. Pairing from the start of y1 and y2′, and the subsequent samples, compute values at each time sample k:

$$r 1 ( k ) = y 1 ( k ) / y {2}^{′} ( k ) ,$$

$$b 1 ( k ) = median ( \{ r 1 ( k - {T}_{w} ) , ... , r 1 ( k + {T}_{w} ) \} )$$

$$n 1 ( k ) = r 1 ( k ) / b 1 ( k )$$

where T_{w} represents a time window large enough to enable the calculation of the patient's baseline glucose levels relative to each time instance k; and
where n represents a baseline-normalized ratio between two sensor glucose values.
f. Any time period within the x1 time series with an n1 value below a predetermined threshold, n_low, may be identified as a dropout. The threshold n_low may be equal to a fixed amount, such as 0.95 for example, or in some embodiments, a variable amount based on an initial value (e.g., 0.95) that increases as a function of the latest sensor noise quality. Other values may be used.
g. Repeat the process by replacing y2=x2 with the next time series (e.g., set y2 equal to x3, x4, . . . , xN−1).

In another embodiment, curve smoothing, dynamic range matching, and time dilation are used to enable comparison of corresponding pre-smoothed time series. The addition of dynamic range matching scales the analyte concentration amplitudes to make comparison easier. For each of the N−1 comparisons (e.g., x1 vs. x2; x1 vs. x3; . . . x1 vs. xN−1), the computer system may perform the following steps.
a. Set y1 equal to the x1 time series and define y2 to be the next time series to compare against (e.g., x2, x3, . . . , or xN−1).
b. Determine the dynamic range of y1 time series by estimating upper (y1u), lower (y1l), and average (y1a) values.
   For example: sort all y1 values, and determine the 5^{th} and 95^{th} percentile values. Set y1u to the 95^{th} percentile value in y1, and set y1l to the 5^{th} percentile value in y1. Set y1a to the 50^{th} percentile value in y1. Other values may be used.
c. Calculate the dynamic range of y2 time series using the same method.
d. Adjust the dynamic range of y2 time series to match that of y1.
For example, compute y2″: =y1a+ [[y2−y2a] [y1u−y2l]/[y2u−y2]]
e. Define a finite set of time dilation (Ta) parameters.
   For example: T_{d}={5/10, 6/10, 7/10, 8/10, 9/10, 1, 10/9, 10/8, 10/7, 10/6, 10/5}
f. For each T_{d} value:
   i. Stretch or compress y2″ by the T_{d} value.
      For example, for T_{d}=5/10=1/2, the time series y2″ is compressed to be half as short as its original. For T_{d}=10/5=2, the time series y2″ is stretched to be twice as long as its original. Other values may be used.
   ii. Compute y2′, where the values are interpolated such that the time series y2′ has the same sample time as y1. A linear interpolation method or any other practicable method of interpolation may be used.
   iii. Compute the cross correlation between y1 and y2′.
   iv. Repeat by using the next T_{d} value in the set.
g. Find the T_{d} value that results in the highest cross-correlation between y1 and y2′. The T_{d} value that results in the highest cross-correlation between y1 and y2′ is the best time dilation adjustment.
h. Pairing from the start of y1 and y2′, and the subsequent samples, compute values at each time sample

$$k : r 1 ( k ) = y 1 ( k ) / y {2}^{′} ( k ) ,$$

$$b 1 ( k ) = median ( \{ r 1 ( k - {T}_{w} ) , ... , r 1 ( k + {T}_{w} ) \} )$$

$$n 1 ( k ) = r 1 ( k ) / b 1 ( k )$$

i. Any time period within the x1 time series with an n1 value below a predetermined threshold, n_low, may be identified as a dropout. The threshold n_low may be equal to a fixed amount, such as 0.95 for example, or in some embodiments, a variable amount based on an initial value (e.g., 0.95) that increases as a function of the latest sensor noise quality. Other values may be used.
j. Repeat the process by replacing y2=x2 with the next time series (e.g., set y2 equal to x3, x4, . . . , xN−1).

In some other embodiments, the process is similar to the two embodiments described above until the point in which a dropout is identified. Instead of identifying a dropout segment whenever any one of the comparator time series (x2, x3, . . . , xN−1) results in an n1 value below a predetermined threshold, a dropout segment is identified whenever any one of the comparator time series results in an n1 value below a predetermined threshold n_low and no other n1 at the same time period is higher than n_high where, for example, n_high equals approximately 1.2. Other values may be used.

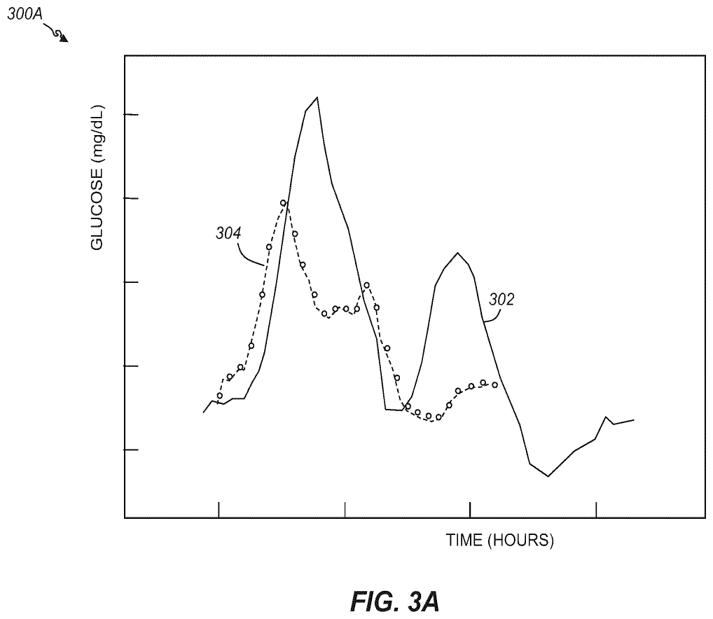

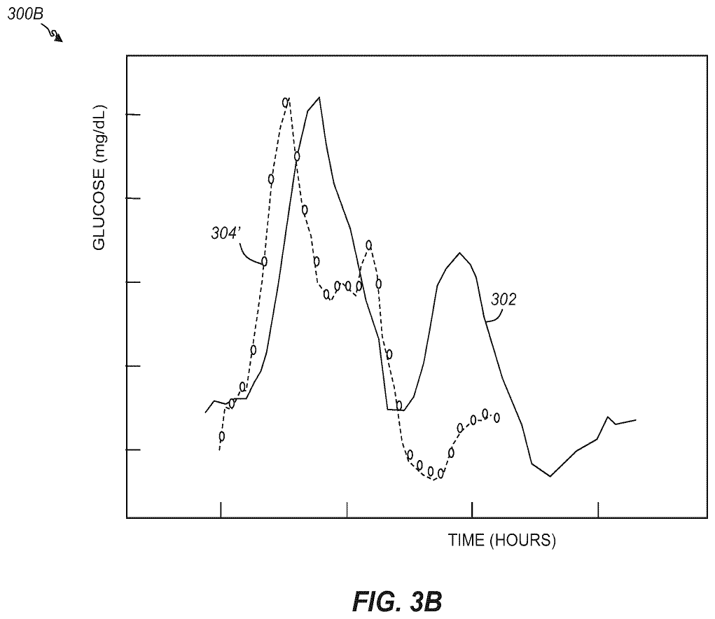

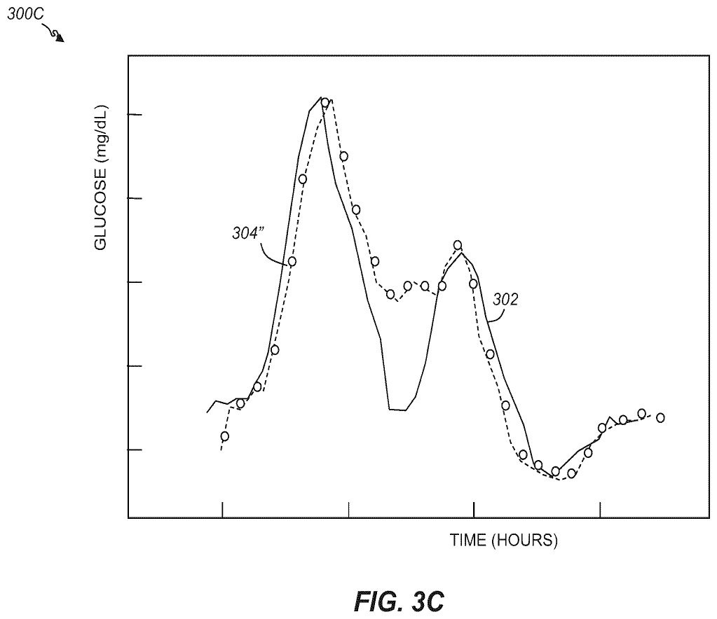

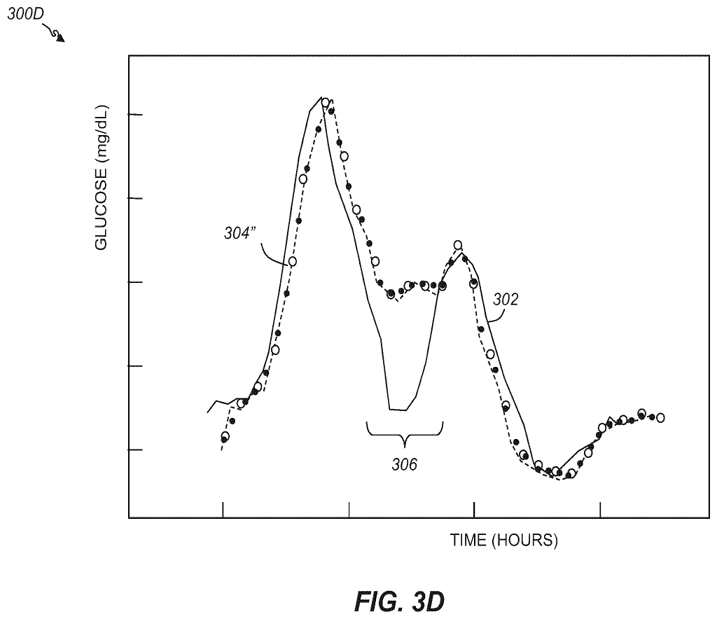

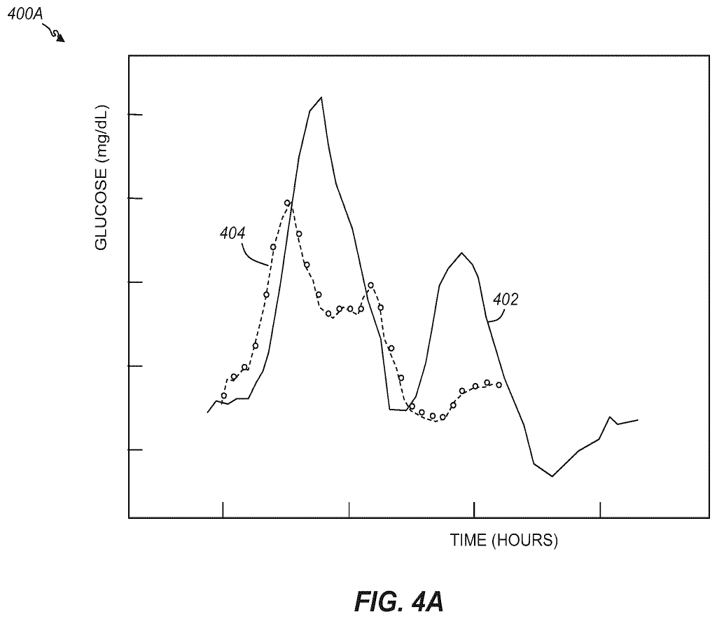

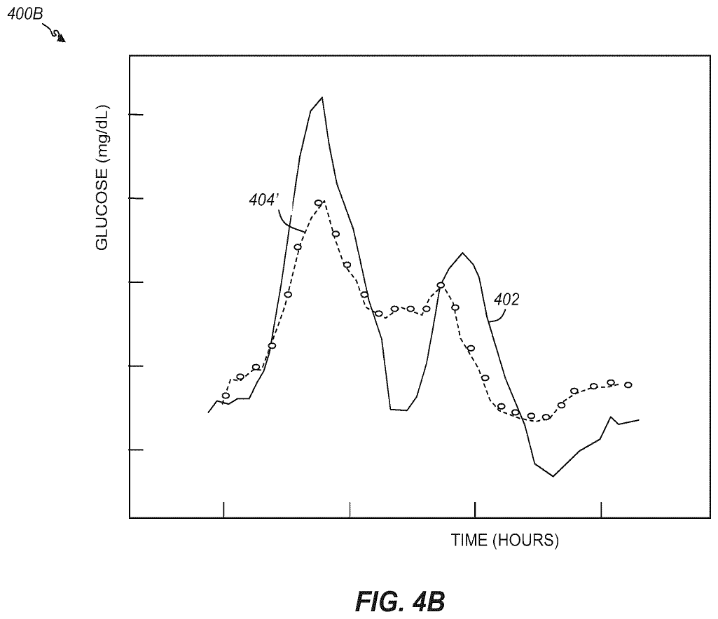

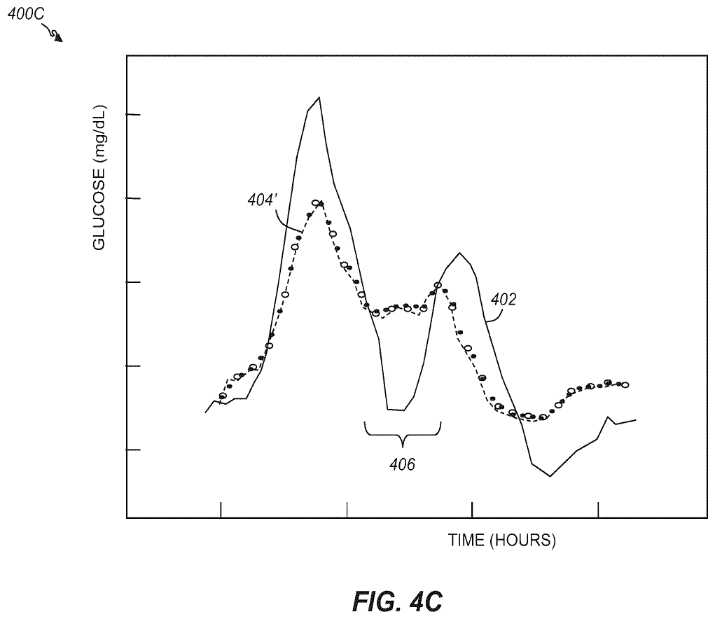

The above processes compare a most recent time series against all available corresponding time series to identify periods within the most recent time series that are dropouts. FIGS. 3A to 3D and 4A to 4C graphically depict simplified embodiments of this process. FIG. 3A depicts a graph 300A of a simple case where only two time series 302, 304 are to be compared to identify dropouts. The plots of the two time series 302, 304 have been smoothed as described above. Comparing the plots 302, 304 in FIG. 3A relative to each other, reveals that the analyte concentration data excursion caused by the event (e.g., a meal) associated with the plot 302 was larger than the analyte concentration data excursion caused by the event associated with the plot 304 (e.g., more carbohydrates were consumed during the meal associated with plot 302 than during the meal associated with plot 304). Thus, a dynamic range scaling operation as described above may be used to stretch plot 304 vertically to facilitate identification of any dropouts. An example of the results of the dynamic range scaling operation on the plot 304 is depicted in FIG. 3B.

Analysis of FIG. 3B, which is a graph 300B, reveals that the repeating event associated with the plot 304 took less time than the event associated with the plot 302. Thus, a time dilation operation as described above may be used to stretch plot 304 laterally to facilitate identification of any dropouts. FIG. 3C is a graph 300C that illustrates an example of the results of the time dilation operation on the plot 304.

FIG. 3D is a graph 300D that represents the results of dynamic range scaling and time dilating of plot 304 so that it best matches (e.g., has the highest cross-correlation with) plot 302. In other words, plot 304″ represents the dynamic range scaled and time dilated version of plot 304 that most closely correlates (e.g., overlaps) with plot 302. With the two plots 302, 304 smoothed, plot 304 dynamic range scaled to plot 304′ and time dilated to plot 304″, the period labeled 306 can easily be identified as a dropout in plot 302. In some embodiments, the computer system may provide the user with a display similar to the graph 300D of FIG. 3D with an indication that the time period 306 represents a dropout.

FIG. 4A depicts a graph 400A of another simple case where only two time series 402, 404 are to be compared to identify dropouts. The plots of the two time series 402, 404 have been smoothed as described above. Comparing the plots 402, 404 in FIG. 4A relative to each other, reveals that the repeating event associated with the plot 404 took less time than the event associated with the plot 402. Thus, a time dilation operation as described above may be used to stretch plot 404 laterally to facilitate identification of any dropouts. FIG. 4B illustrates an example of the results of the time dilation operation on the plot 404.

From the graph 400B in FIG. 4B, a dropout can be identified as described above, without having to perform dynamic range scaling operation. Thus, FIG. 4C is a graph 400C that represents the results of time dilating plot 404 so that it best matches (e.g., has the highest cross-correlation with) plot 402. In other words, plot 404′ represents the time dilated version of plot 404 that most closely correlates (e.g., overlaps) with plot 402. With the two plots 402, 404 smoothed and plot 404 time dilated to plot 404′, the period labeled 406 can easily be identified as dropout in plot 402. In some embodiments, the computer system may provide the user with a display similar to the graph 400C of FIG. 4C with an indication that the time period 406 represents a dropout.

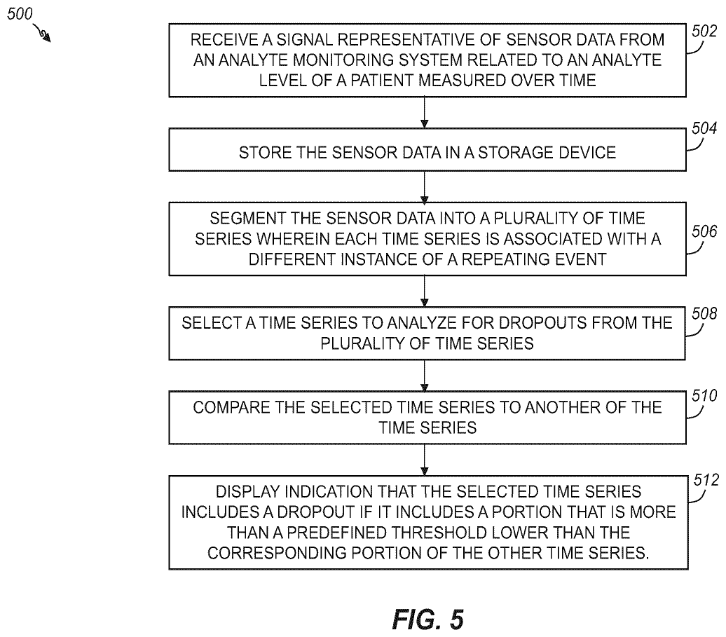

Turning now to FIG. 5, a flow chart illustrating an example method 500 of the present invention is provided. In some embodiments, a signal representative of sensor data is received by the computer system from an analyte monitoring system, for example, a CGM (502). The sensor data may include a historical record of an analyte level of a patient measured over time. The sensor data is stored in a storage device of the computer system (504). In some embodiments, the sensor data is segmented into a plurality of time series (506). Each time series is associated with a different instance of a repeating event. In some embodiments, the sensor data may be smoothed as described above, to facilitate the segmenting. In some embodiments, a meal start time may be identified for each of the time series to facilitate identifying the start and end of the time series. The meal start time may be manually entered (e.g., based on information provided by the patient) or automatically determined using estimating software as mentioned above.

Once one of the time series is selected to be analyzed for dropouts (508), the selected time series may be compared to at least one of the other time series (510). In some embodiments, time dilation and dynamic range matching techniques as described above may be performed on the time series used for comparison to aid in correlating the two time series and to compensate for different lengths of time and different magnitudes of the associated events. The computer system determines whether the selected time series includes a portion that is more than a predefined threshold lower than a corresponding portion of the time series used for comparison (512). The predefined threshold may be selected as described above. If there is a portion of the selected time series that is lower than the comparison time series by more than the threshold amount (e.g., a negative glucose level difference larger than x amount), the computer system displays an indication that the selected time series includes a dropout. In some embodiments, the sensor data that represents the dropout may be removed, set to a zero value, or otherwise marked as invalid data. In some embodiments, the dropout sensor data may be replaced with interpolated data.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

What is claimed is:

1. A method, comprising:
receiving sensor data from an analyte sensor configured to be positioned at least in part in contact with a fluid under a skin layer, the sensor data corresponding to a monitored analyte level of the fluid;
segmenting the sensor data into a plurality of time segments, the plurality of time segments including a first segment comprising a first instance, and a second segment comprising a second instance;
applying a time dilation and correlation operation to the sensor data of the first segment to obtain a time dilated and correlated first segment, wherein the time dilation and correlation operation comprises:
performing a first one of a time dilation operation or a correlation operation to the sensor data of the first segment,
the time dilation operation comprising dilating the sensor data of the first segment to obtain the time dilated first segment, wherein the sensor data of the time dilated first segment over a first period of time corresponds to the sensor data of the second segment over a second period of time, and
the correlation operation comprising correlating the sensor data of the first segment over the first period of time to the sensor data of the second segment over the second period of time to obtain the correlated first segment;
applying a second one of the time dilation operation or the correlation operation to the time dilated first segment or the correlated first segment to obtain the time dilated and correlated first segment; and
determining that the first segment includes a dropout when the time dilated and correlated first segment over the first period of time differs by more than a threshold from the sensor data of the second segment over the second period of time.
